(19) United States
(12) Patent Application Publication
(10) Pub. No.: US 2011/0257748 A1
(43) Pub. Date: Oct. 20, 2011

(54) ARTIFICIAL SPINAL IMPLANT

(76) Inventor: JUNG-TUNG LIU, Taichung City (TW)

(21) Appl. No.: 12/761,205

(22) Filed: Apr. 15, 2010

(51) Int. Cl. A61F 2/44

(52) U.S. Cl. 623/17.16

(57) ABSTRACT

One embodiment of an artificial spinal implant includes an upper positioning unit comprising a first plate member; a lower positioning unit comprising a second plate member parallel to the first plate member; and an elastic member comprising a bent first elastic half and a bent, opposite, integral second elastic half wherein upper and lower ends of each of the first and second elastic halves are formed with the upper and lower positioning units respectively. The artificial spinal implant has the benefits of not contacting the critical elements of the nervous system housed in the spinal column in surgery and dispersing load on the artificial spinal implant.

### BACKGROUND OF THE INVENTION

1. Field of Invention

The invention relates to spinal implants and more particularly to such an artificial spinal implant with improved characteristics.

2. Description of Related Art

Vertebra is also called spine for supporting human body. For spine, from top to bottom, it consists of 7 cervical vertebrae, 12 thoracic vertebrae, 5 lumbar vertebrae, sacrum, and coccyx. They function to support body and protect the nervous system. Intervertebral disc functions as a buffer between components of the vertebra so that spine can flex.

Genetic or developmental irregularities, trauma, chronic stress, and tumors, however, can result in spinal pathologies which either limit the range of motion or threaten the nervous system housed in the spinal column. In many orthopedic surgeries, artificial spinal implants are inserted into the spinal column to support. This is a great contribution to the advancement of medical technology.

However, for patients having degenerative disc disease, the artificial spinal implants do little to relieve localized stress on the spine. Degenerative disc disease often occurs on the lumbar vertebrae. Spinal canal can be pressed to become narrow when stress concentrates on the lumbar vertebrae. This in turn can stress the nervous system. While artificial spinal implants may provide support to the spinal column, the human body may become less flexible due to the rigid nature of the artificial spinal implants.

Recently, artificial discs have been introduced in various orthopedic surgical procedures for cure degenerative disc disease. Artificial discs are classified as hydraulic, elastic, hydrostatic, and wear types. The artificial discs can support the recessed spinal canal so that the damaged disc can be cured.

A typical artificial spinal implant is shown in FIG. 1 and comprises an upper disc member, a lower disc member, and an intermediate body having a top convex portion for matingly engaging a bottom recess of the upper disc member, and a bottom convex portion for matingly engaging a top recess of the lower disc member. As a result, a great deal of strength is provided to support the vertebrae by the artificial spinal implant. The generally spherical contacts between the intermediate body and the upper disc member and between the intermediate body and the lower disc member thus make the artificial spinal implant to be capable of bending, extending and rotating.

However, no limit or stop mechanism is provided to limit the above movements to a desired range. Thus, undesired excess movements may occur. To the worse, dislocation due to wear may occur.

U.S. Pat. No. 7,465,317 discloses an artificial spinal implant. However, it is a mono structural member. Further, it is required to implant from the front of the body. This can reduce surgical safety and is difficult of performing. Thus, the need for improvement still exists.

### SUMMARY OF THE INVENTION

It is therefore one object of the invention to provide an artificial spinal implant comprising an upper positioning unit comprising a first plate member; a lower positioning unit comprising a second plate member parallel to the first plate member; and an elastic member comprising a bent first elastic half and a bent, opposite, integral second elastic half wherein upper and lower ends of each of the first and second elastic halves are formed with the upper and lower positioning units respectively.

It is another object of the invention to provide an artificial spinal implant comprising an upper positioning unit comprising a first plate member; a lower positioning unit comprising a second plate member parallel to the first plate member; and at least one elastic member each comprising a bent first elastic half and a bent, opposite, separate second elastic half, wherein upper and lower ends of each of the first and second elastic halves are formed with the upper and lower positioning units respectively; and wherein an intermediate portion of each of the first and second elastic halves is formed as a pivot with an upper inclined portion of each of the first and second elastic halves extending at an acute angle with respect to a lower inclined portion thereof.

It is still another object of the invention to provide an artificial spinal implant comprising an upper positioning unit comprising a first plate member; a lower positioning unit comprising a second plate member parallel to the first plate member; and at least one elastic member each comprising a bent first elastic half and a bent, opposite, offset, integral second elastic half wherein upper and lower ends of each of the first and second elastic halves are formed with the upper and lower positioning units respectively.

Preferably, a top surface of the first plate member is formed with a plurality of spikes and a bottom surface of the second plate member is formed with a plurality of spikes; and the elastic member is formed of metal wire.

Preferably, an intermediate portion of each of the first and second elastic halves is formed as a pivot with an upper inclined portion of each of the first and second elastic halves extending at an acute angle with respect to a lower inclined portion thereof.

The above and other objects, features and advantages of the invention will become apparent from the following detailed description taken with the accompanying drawings.

### BRIEF DESCRIPTION OF THE DRAWINGS

FIG. 1 is an exploded view of a typical artificial spinal implant;

FIG. 2 is a side view of an artificial spinal implant according to a first preferred embodiment of the invention;

FIG. 3 is a front view of the artificial spinal implant of FIG. 2;

FIG. 4 is a side view of the artificial spinal implants of FIG. 2 fastened between two vertebrae in a spinal column;

FIG. 5 is a front view of FIG. 4;

FIG. 6 is a side view showing another configuration of the elastic member;

FIG. 7 is a side view showing still another configuration of the elastic member;

FIG. 8 is a perspective view of an artificial spinal implant according to a second preferred embodiment of the invention;

FIG. 9 is a side view of the artificial spinal implant of FIG. 8;

FIG. 10 is a front view of the artificial spinal implant of FIG. 8;

FIG. 11 is a front view of the artificial spinal implants of FIG. 8 fastened between two vertebrae in a spinal column;

FIG. 12 is a side view of the artificial spinal implants of FIG. 9 fastened between two vertebrae in a spinal column;

FIG. 13 is a side view of an artificial spinal implant according to a third preferred embodiment of the invention;

FIG. 14 is a sectional view taken along line X-X of FIG. 13;

FIG. 15 is a side view of an artificial spinal implant according to a fourth preferred embodiment of the invention; and

FIG. 16 is a sectional view taken along line Y-Y of FIG. 15.

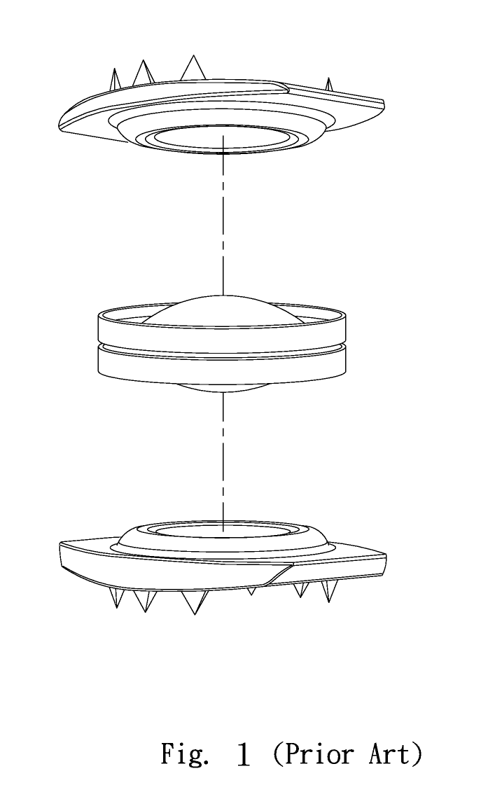

### DETAILED DESCRIPTION OF THE INVENTION

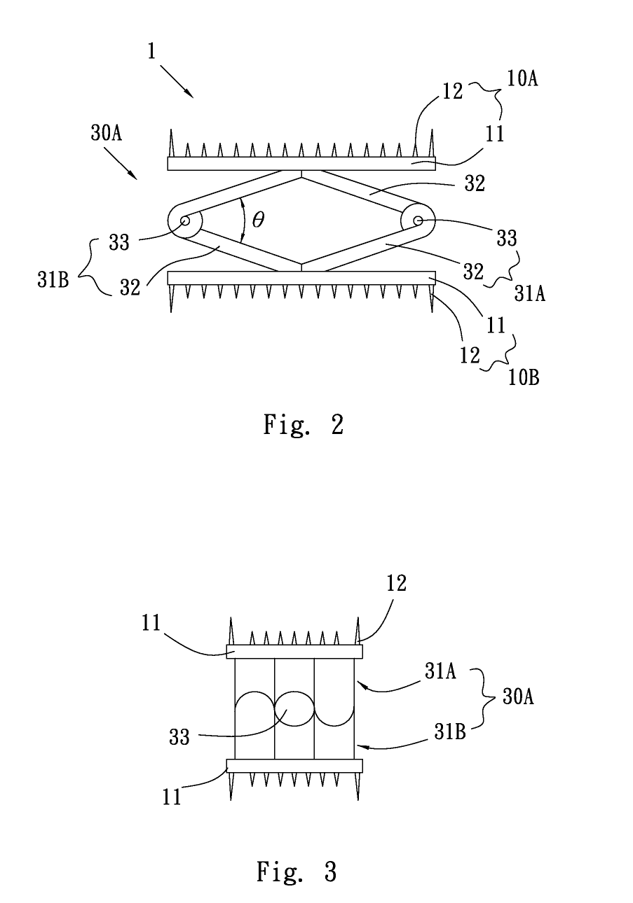

Referring to FIGS. 2 to 7, an artificial spinal implant 1 in accordance with a first preferred embodiment of the invention is made of titanium alloy and comprises an upper positioning unit 10A, a lower positioning unit 10B, and an elastic member 30A. Each component will be discussed in detail below.

Each of the upper positioning unit 10A and the lower positioning unit 10B comprises parallel upper plate member 11 and lower plate member 11. A top surface of the upper plate member 11 is formed with a plurality of spikes 12 and a bottom surface of the lower plate member 11 is formed with a plurality of spikes 12.

The elastic member 30A comprises a first elastic half 31A and an opposite, integral second elastic half 31B each being formed of elastic wire made of titanium alloy and bent. Each of the first elastic half 31A and the second elastic half 31B comprises upper and lower inclined members 32 joined at an intermediate pivot 33. The upper inclined member 32 is formed with the upper positioning unit 10A and the lower inclined member 32 is formed with the lower positioning unit 10B. The upper inclined member 32 extends at an angle θ with respect to the lower inclined member 32.

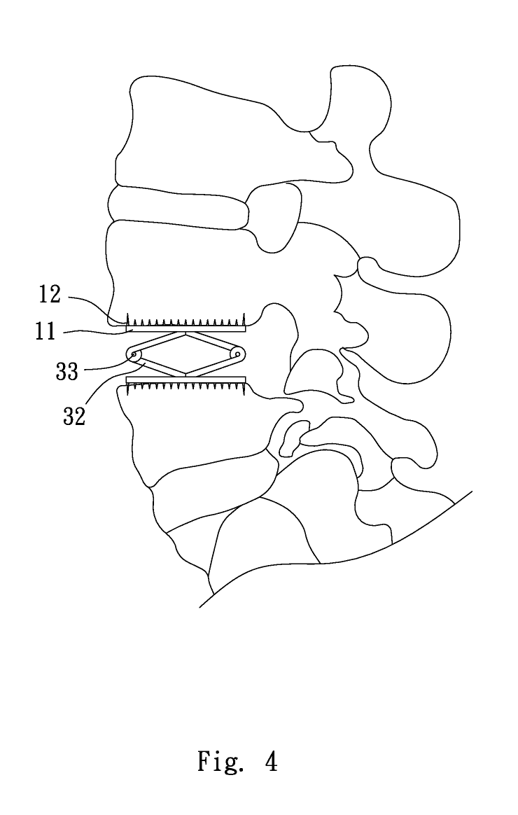

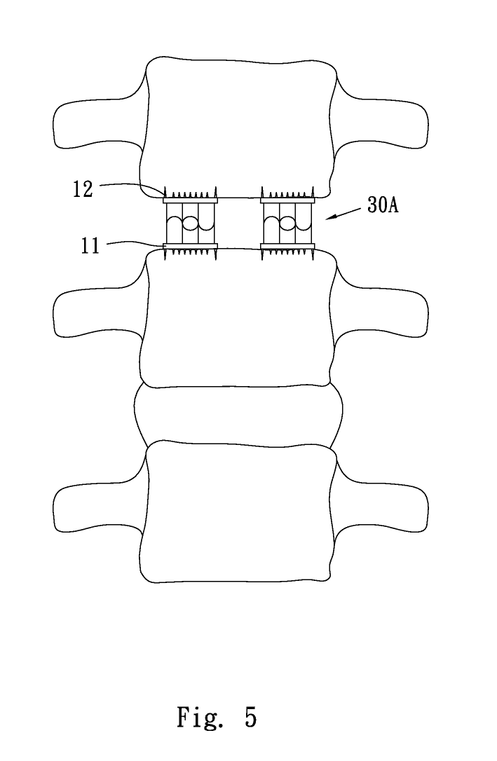

As show in FIGS. 4 and 5, two artificial spinal implants of the invention are sequentially implanted between two vertebrae in a spinal column from rear of the human body. This has the benefits of not contacting the critical elements of the nervous system housed in the spinal column in surgery and dispersing load on the artificial spinal implants, i.e., the elastic member 30A being capable of absorbing force exerted from both upward and downward directions.

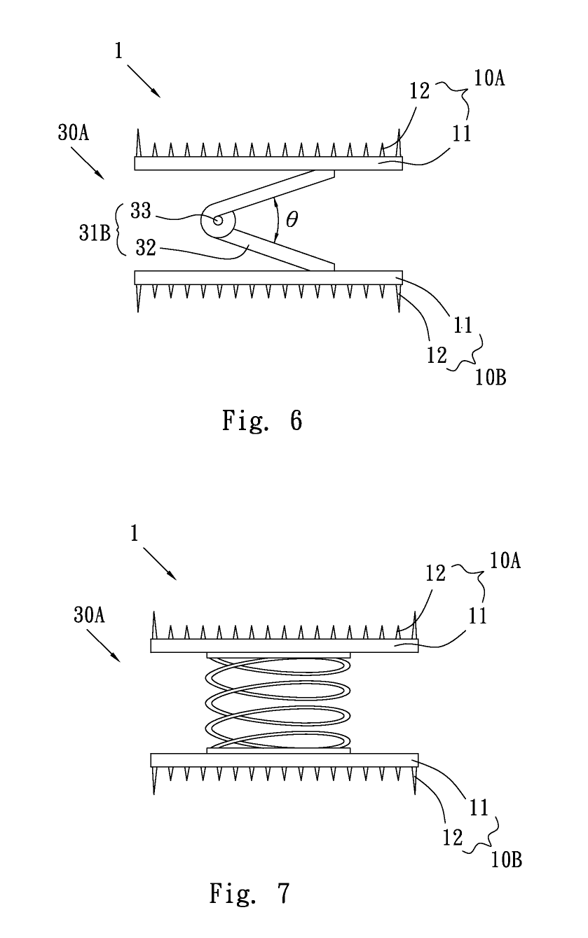

As shown in FIG. 6, alternatively the elastic member 30A is implemented as only one elastic half 31B in which the upper inclined member 32 extends at an angle θ with respect to the lower inclined member 32. As shown in FIG. 7, still alternatively the elastic member 30A is implemented as a helical spring fastened between the upper positioning unit 10A and the lower positioning unit 10B.

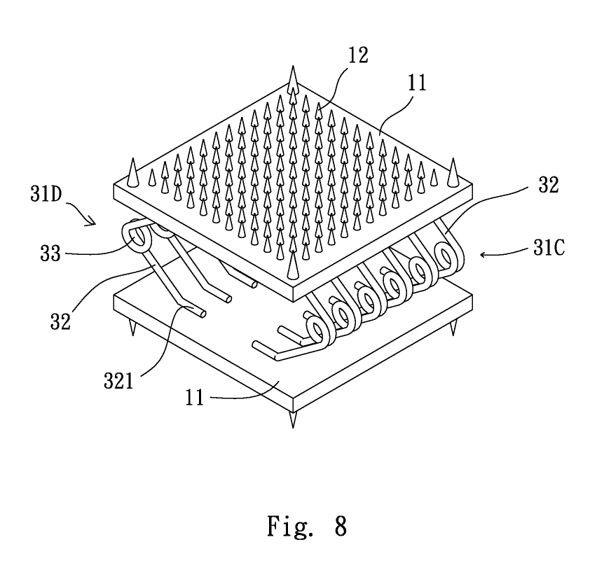

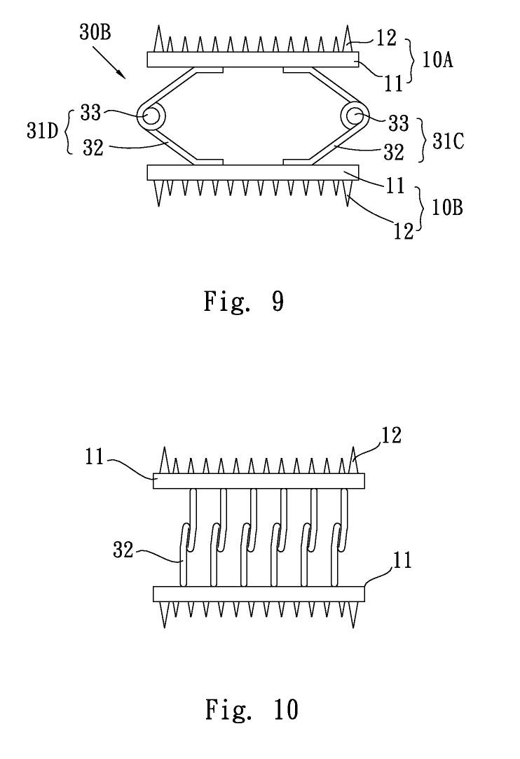

Referring to FIGS. 8 to 12, an artificial spinal implant in accordance with a second preferred embodiment of the invention comprises an upper positioning unit 10A, a lower positioning unit 10B, and a plurality of parallel elastic members 30B each formed of elastic wire made of titanium alloy and bent. Each component will be discussed in detail below.

Each of the upper positioning unit 10A and the lower positioning unit 10B comprises parallel upper plate member 11 and lower plate member 11. A top surface of the upper plate member 11 is formed with a plurality of spikes 12 and a bottom surface of the lower plate member 11 is formed with a plurality of spikes 12.

The elastic member 30B comprises a first elastic half 31C and an opposite, separate second elastic half 31D each being bent. Each of the first elastic half 31C and the second elastic half 31D comprises upper and lower inclined members 32 joined at an intermediate pivot 33. The upper inclined member 32 is formed with the upper positioning unit 10A and the lower inclined member 32 is formed with the lower positioning unit 10B. The upper inclined member 32 extends at an angle θ with respect to the lower inclined member 32.

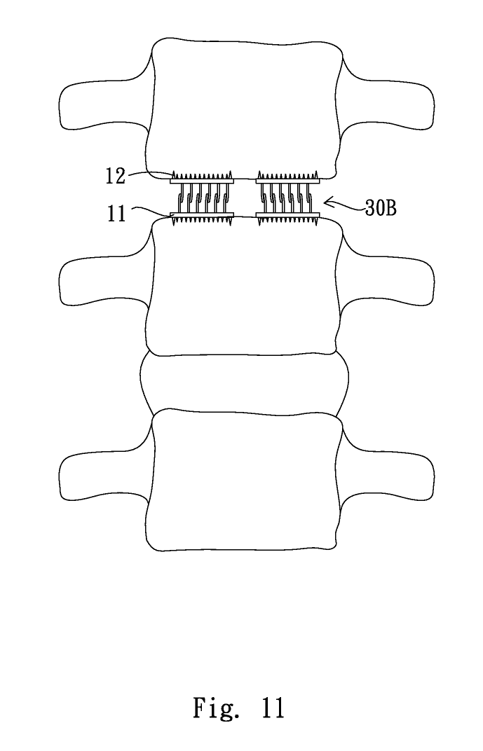

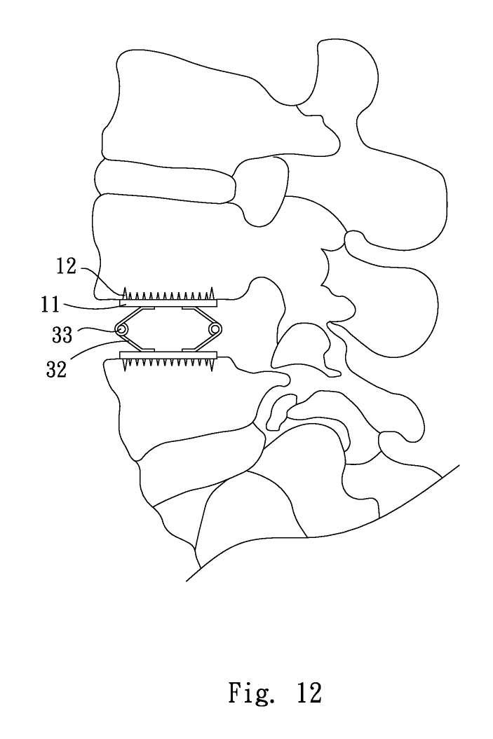

As show in FIGS. 11 and 12, two artificial spinal implants of the invention are sequentially implanted between two vertebrae in a spinal column from rear of the human body. This also has the benefits of not contacting the critical elements of the nervous system housed in the spinal column in surgery and dispersing load on the artificial spinal implants, i.e., the elastic members 30B being capable of absorbing force exerted from both upward and downward directions.

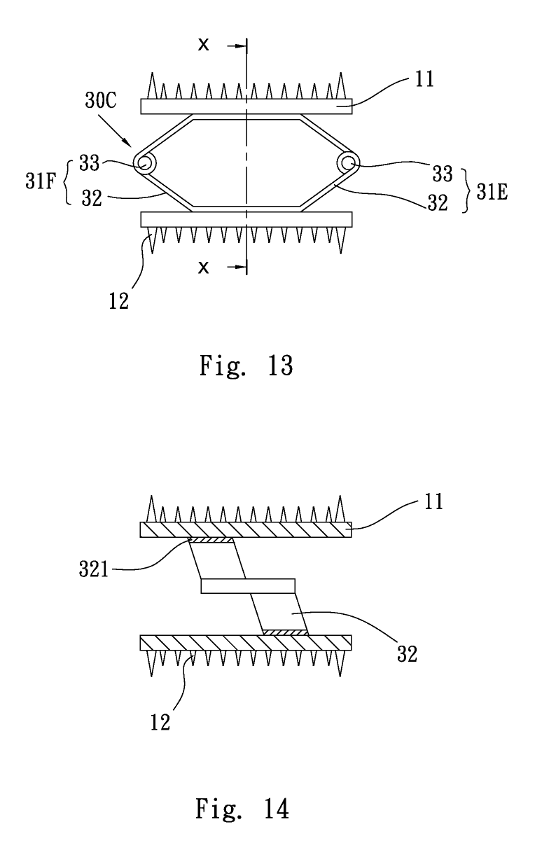

Referring to FIGS. 13 and 14, an artificial spinal implant in accordance with a third preferred embodiment of the invention is characterized below.

An elastic member 30C is formed of elastic plate made of titanium alloy and bent. The elastic member 30C comprises a first elastic half 31E and an opposite, offset, integral second elastic half 31F. Each of the first elastic half 31E and the second elastic half 31F comprises upper and lower inclined members 32 joined at an intermediate pivot 33. The upper or lower inclined member 32 is formed with the plate member 11 having spikes 12 on outer surface. The upper inclined member 32 extends at an angle θ with respect to the lower inclined member 32.

This also has the benefits of not contacting the critical elements of the nervous system housed in the spinal column in surgery and dispersing load on the artificial spinal implants, i.e., the elastic member 30C being capable of absorbing force exerted from both upward and downward directions.

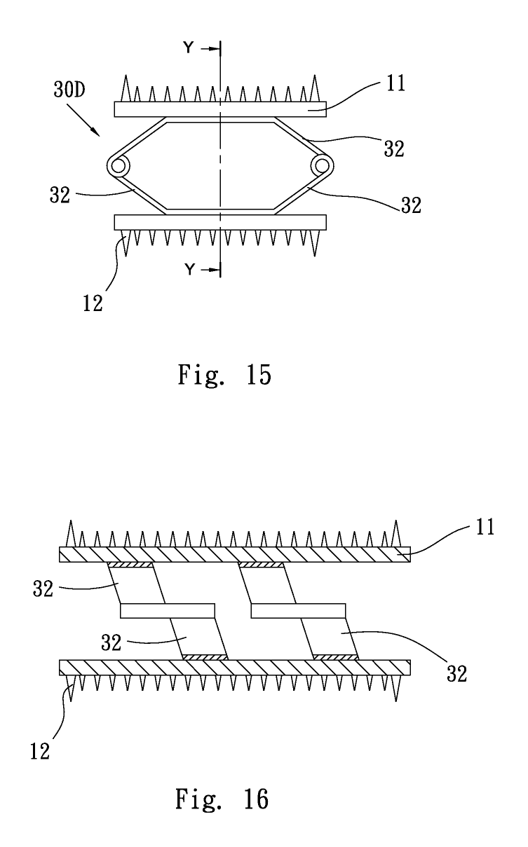

Referring to FIGS. 15 and 16, an artificial spinal implant in accordance with a fourth preferred embodiment of the invention is characterized below.

Each of two parallel elastic members 30D is formed of elastic plate made of titanium alloy and bent. Each of the elastic members 30D comprises a first elastic half and an opposite, offset, integral second elastic half each being bent. Each of the first elastic half and the second elastic half comprises upper and lower inclined members 32 joined at an intermediate pivot. The upper or lower inclined member 32 is formed with the plate member 11 having spikes 12 on outer surface. The upper inclined member 32 extends at an angle θ with respect to the lower inclined member 32.

This also has the benefits of not contacting the critical elements of the nervous system housed in the spinal column in surgery and dispersing load on the artificial spinal implants, i.e., the elastic members 30D being capable of absorbing force exerted from both upward and downward directions.

While the invention herein disclosed has been described by means of specific embodiments, numerous modifications and variations could be made thereto by those skilled in the art without departing from the scope and spirit of the invention set forth in the claims.

What is claimed is:

1. An artificial spinal implant comprising:
an upper positioning unit comprising a first plate member;
a lower positioning unit comprising a second plate member parallel to the first plate member; and
an elastic member comprising a bent first elastic half and a bent, opposite, integral second elastic half wherein upper and lower ends of each of the first and second elastic halves are formed with the upper and lower positioning units respectively.

2. The artificial spinal implant of claim 1, wherein a top surface of the first plate member is formed with a plurality of spikes and a bottom surface of the second plate member is formed with a plurality of spikes; and wherein the elastic member is formed of metal wire.

3. The artificial spinal implant of claim 1, wherein an intermediate portion of each of the first and second elastic halves is formed as a pivot with an upper inclined portion of each of the first and second elastic halves extending at an acute angle with respect to a lower inclined portion thereof.

4. An artificial spinal implant comprising:
an upper positioning unit comprising a first plate member;
a lower positioning unit comprising a second plate member parallel to the first plate member; and
at least one elastic member each comprising a bent first elastic half and a bent, opposite, separate second elastic half,
wherein upper and lower ends of each of the first and second elastic halves are formed with the upper and lower positioning units respectively; and
wherein an intermediate portion of each of the first and second elastic halves is formed as a pivot with an upper inclined portion of each of the first and second elastic halves extending at an acute angle with respect to a lower inclined portion thereof.

5. The artificial spinal implant of claim 4, wherein a top surface of the first plate member is formed with a plurality of spikes and a bottom surface of the second plate member is formed with a plurality of spikes; and wherein the elastic member is formed of metal wire.

6. An artificial spinal implant comprising:
an upper positioning unit comprising a first plate member;
a lower positioning unit comprising a second plate member parallel to the first plate member; and
at least one elastic element each comprising a bent first elastic half and a bent, opposite, offset, integral second elastic half wherein upper and lower ends of each of the first and second elastic halves are formed with the upper and lower positioning units respectively.

7. The artificial spinal implant of claim 6, wherein a top surface of the first plate member is formed with a plurality of spikes and a bottom surface of the second plate member is formed with a plurality of spikes; and wherein each elastic element is formed of metal plate.

8. The artificial spinal implant of claim 6, wherein an intermediate portion of each of the first and second elastic halves is formed as a pivot with an upper inclined portion of each of the first and second elastic halves extending at an acute angle with respect to a lower inclined portion thereof.

9. The artificial spinal implant of claim 6, where the at least one elastic element comprises two parallel elastic elements.
